(19) United States
(12) Patent Application Publication
(10) Pub. No.: US 2007/0287882 A1
(43) Pub. Date: Dec. 13, 2007

(54) Method and device for mood improvement

(76) Inventor: Brent Ellis Logan, Edmonds, WA (US)

Correspondence Address: Brent E. Logan, 1004 Daley Street, Edmonds, WA, 98020, US

(21) Appl. No.: 11/807,428

(22) Filed: May 29, 2007

Related U.S. Application Data
(60) Provisional application No. 60/809,571, filed on Jun. 1, 2006.

(51) Int. Cl. A61M 21/00

(52) U.S. Cl. 600/26

(57) ABSTRACT

The present invention provides a method for alleviating negative moods by physically raising the lateral ends of the lips to signal their facial muscles of an elevated position, which information is then conveyed through the nervous system to positivistic centers in the brain, thereby inducing an improved psychodynamic. Various means utilizing noninjurious and nontoxic materials for achieving this effect include hooks that lift the opposing corners of the lips, and are attached to an adjustable headband or earpieces, alternatively a semirigid rod which curves upward, fitting inside the lower lip, with saddles at its ends for elevating the lateral extremities of the lips, additionally a flexible lower tooth covering with upwardly protruding spurs on either side to attain the same purpose. Not an exerciser for the mouth, facial muscles, or neck, this device in its several configurations is contraindicated as a diagnostic or therapeutic aid, without the intent of medical application.

### CROSS-REFERENCE TO RELATED APPLICATIONS

This application claims the benefit of U. S. Provisional Patent Application No. 60/809,571 having a filing date of Jun. 1, 2006, the primary contents of which are hereby incorporate ( by reference.

### STATEMENT REGARDING FEDERALLY SPONSORED RESEARCH OR DEVELOPMENT

Not Applicable

### REFERENCE TO SEQUENCE LISTING, A TABLE, OR A COMPUTER LISTING COMPACT DISK APPENDIX

Not Applicable

### BACKGROUND OF THE INVENTION

Because for homeostatic survival benefit depressed states are never sought by individuals excepting those with an acute psychological condition, pleasure may be presumed an optimal human goal, the normative bond which holds families and societies together. Nonetheless, contemporary life places intense burdens upon everyone, from the effects of demographic overload hence workplace competition to an information glut which makes choices uncertain, sometimes confused. The result of these stressors can be emotional dysfunction or volatile behavioral swings for which the typical palliatives such as medications, tobacco, alcohol, and materialism provide little relief, frequently becoming addictive.

While the connection between facial mien and mood may seem obvious, it became the subject of major scientific examination with publication by Charles Darwin of The Expression of the Emotions in Man and Animals. From a growing knowledge about how muscles by way of nerves trigger chemical activity in the brain which evokes emotional responses, it was inevitable that this information would seek practical use, accounting for Anatomy of an Illness, where Norman Cousins cited the curative powers of humor, also underscored in Laughter: A Scientific Investigation, by Robert Provine. Clinically, such an approach became known as psychoneuroimmunology, with the work of Fritz Strick, Leonard Martin, and Sabine Stepper conducting a basic though telling investigation where subjects clenched a pencil in their teeth but without lips touching it, thereby forcing a smile, for 15 seconds, the outcome appreciably improving mood. More recent replications of this classic experiment reveal that in only 5 seconds, 75 percent of volunteers expressed emotional benefit. These results are reinforced by a study of twins from David Lykken and Auke Tellegen which discounts genetics as a leading factor in the ability to be happy.

What this background demonstrates is that if the brain initiates thoughts or feelings which are manifested in smiles or frowns, the reverse process holds. A physical upturn at the edges of the lips can evoke the mental requirements for happiness, the extent of which depends upon length of the stimulus application and individual psychodynamics.

As listed in the U. S. Patent Documents above, several devices for exercising facial muscles are known to the art, but none is intended or designed for mood improvement by means of effecting a smile by physically elevating the lateral extremities of the lips.

### BRIEF SUMMARY OF THE INVENTION

This nonmedical innovation is a safe, effective, unique, and lowcost means for alleviating negative emotions along with the deleterious behaviors which typically result therefrom, including dependency upon often addictive stimulants. Conceived by the inventor in various configurations, the purpose of the device is to physically induce a smile that from altering facial muscles attending the opposing corners of the lips, notably the zygomaticus, sends nerve signals to brain centers responsible for positivistic feelings. Duration of use depends upon degree of the emotional state, availability of time, and degree as well as extent of the beneficial effect from this device. Minor alteration of a negative feeling can be achieved in a few minutes, then reapplied should that original state recur. The uplift in mood can last hours, while a major depressive condition is addressed over longer periods, as determined by the user, even overnight. Regular employment may elicit a permanent sense of wellbeing with corresponding behavioral improvement. If extensive reliance upon this device occurs, occasional repositioning avoids lip or gum soreness. The scientific evidence for response pattern activation modifying psychological inhibition is substantial, with synthetic facial alteration demonstrating that if the brain initiates thoughts or responds to feelings which are manifested in smiles, the reverse process holds irrespective of actual mood once an artificial countermeasure is initiated.

One embodiment of the invention is comprised of two pliable hooks fitting the lateral extremities of the lips, and connected by a stretchable or otherwise adjustable headband such as thin metal or plastic to accommodate cranial dimensions and maintain sufficient tension so the opposing lip corners are elevated, or utilizing separate earpieces of stretchable material or which can be similarly adjusted for size and lip pressure. The band itself is not sufficiently wide as to cause flattening of hairstyles or create cranial warmth, and while rigid enough for retaining shape the surface of the ergonomic hooks should be soft, noninjurious and nontoxic to the mouth, lips, tongue, gums, or teeth. Although stretchable material such as elastic may be utilized, a sliding pressure clip, snap closure, velcro, or similar fastener on the headband or earpieces composed of other noninjurious surfaces and nontoxic materials can also permit accommodation to any head size as well as exert adequate lifting of the lateral lip ends.

Another version is a semirigid round or oval rod curving upward with fixed saddles at either end that raise the lateral extremities of the lips, and which can be produced in different sizes or cut to accommodate the mouth width of the user, fitting inside the lower lip and in front of the teeth, with detachable saddles at either end that elevate both lips at the opposing corners. A third alternative constitutes a flexible lower dental plate which can be produced in different sizes with fixed or adjustable upwardly protruding spurs at either end that raise the lateral extremities of the lips, or alternatively cut to conform with the mouth width of the user, fitting over the teeth, with upwardly protruding spur caps positioned at the opposing ends. Both latter variants, like the first, would have all components directly in contact with the mouth, lips, gums, tongue, teeth, skin, head, or hair composed of noninjurious surfaces and nontoxic materials.

### BRIEF DESCRIPTION OF THE SEVERAL VIEWS OF THE DRAWINGS

The unique features that are characteristic of the present invention are set forth in the appended claims. However, the preferred embodiments of the invention, together with further objects and attendant advantages, are best understood by reference to the following detailed description taken in connection with the accompanying drawings in which:

FIG. 1 is a frontal perspective view of the device positioned on a head depicting two hooks at the lateral lip extremities, and a headband fastened to said hooks elevating same.

FIG. 1A is a ¾ frontal perspective view of the device alone showing two lip hooks connected to a headband in two sections fastened where they meet by an adjustable clip, snap closure, velcro, or other such means.

FIG. 1 B is a ¾ perspective view of the device illustrating a stretchable or adjustable band positioned on a head, and affixed to a single hook raising the right lip at its end.

FIG. 1C is a ¾ perspective viewed from the right of the device by itself portraying two lip hooks attached to a headband in two sections fastened where they meet by an adjustable clip, snap, velcro, or other such means.

FIG. 1D is a left side perspective view of the device alone depicting the left lip hook connected to a headband in two sections attached where they meet by an adjustable clip, snap closure, velcro, or other such means.

FIG. 1E is a full frontal perspective view of the device by itself illustrating two lip hooks affixed to a headband in two sections attached where they meet by an adjustable clip, snap closure, velcro, or other such means.

FIG. 2 is a frontal perspective view of the device on a head showing the outside portions of two removable saddles positioned at the opposing corners of the lips, each saddle capping the end of an upwardly curved rod inside the lower lip, thereby raising the lateral extremities of the lips.

FIG. 2A is a frontal perspective view of the device alone depicting two detachable saddles capped to the ends of an upwardly curved rod which fits inside the lower lip, thereby raising the opposing corners of the lips.

FIG. 3 is a frontal perspective view of the device on a head portraying the exposed portions of two adjustable and upwardly positioned spurs attached to a flexible lower dental plate, which can be positioned at and thereby elevate the opposing corners of the lips.

FIG. 3A is a frontal perspective view of the device by itself illustrating a flexible lower dental plate with two adjustable and upwardly protruding spurs which can be positioned at and thereby lift the opposing corners of the lips.

### DETAILED DESCRIPTION OF THE INVENTION

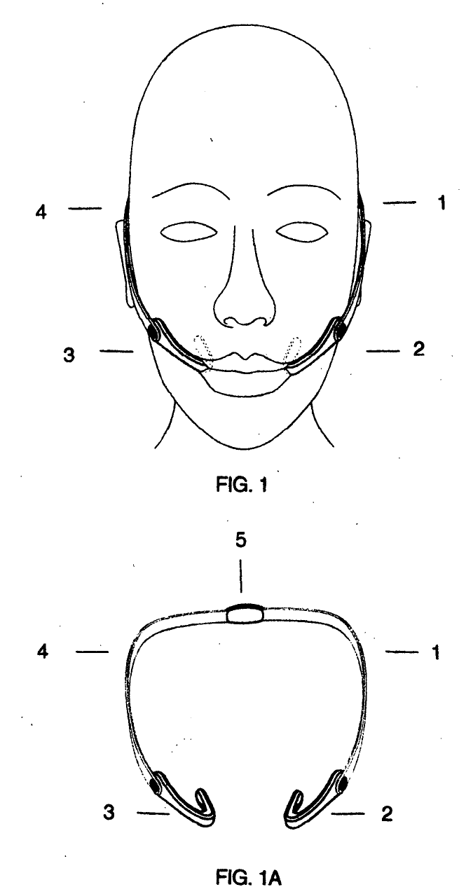

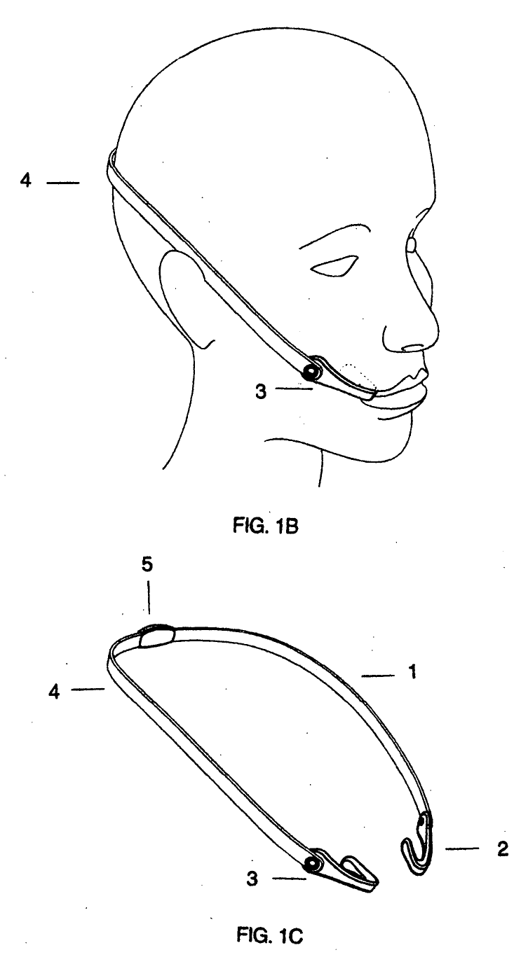

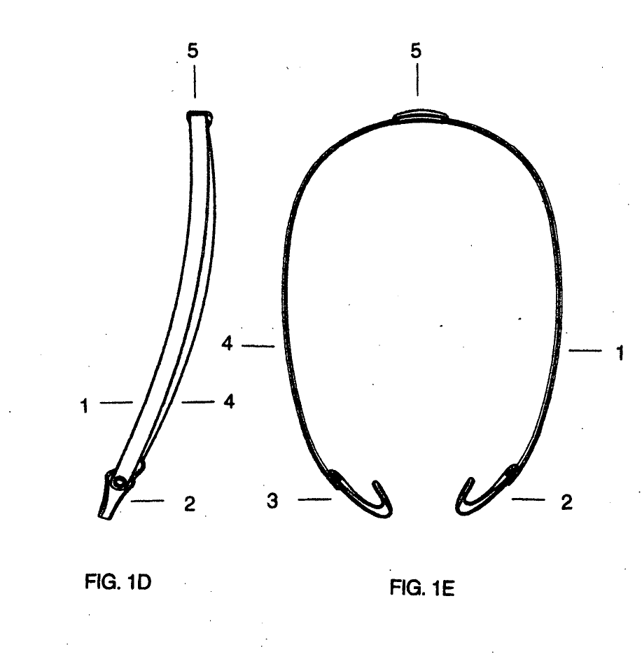

Referring to FIG. 1, one embodiment of the present device in frontal perspective view illustrates on a head the left and right sides, 1 and 4, respectively, of a headband connected to a left and right hook, 2 and 3, respectively, positioned in the opposing corners of the lips, which from the tension of the headband are raised upward into a smile. FIG.1A represents a ¾ frontal perspective view of this same device independent of a head, with the left and right sides, 1 and 4, respectively, of a headband connected to a left and right hook, 2 and 3, respectively, positioned at and thereby elevating the opposing corners of the lips. If not employing elastic, rubber, or other stretchable material for the headband, its left and right halves are attached where they meet by an adjustable clip, snap closure, velcro, or other such means, 5. FIG. 1B depicts a ¾ perspective view of this same embodiment on a head, with the right side of the headband, 4, connected to a right hook, 3, positioned in the right lateral end of the lip. Shown in FIG. 1C is the device independent of a head, viewed in ¾ perspective, where 1 and 4 represent the left and right sides, respectively, of a headband in two halves fastened together by an adjustable clip, snap closure, velcro, or other such means, 5, said headband connected to a left and right hook, 2 and 3, respectively, positioned at and thereby raising the opposing corners of the lips. FIG. 1D portrays a left side perspective view of this device alone, with the left lip hook, 2, attached to the left half, 1, of a headband which is connected by an adjustable clip, snap closure, velcro, or other such means, 5, to its right half, 4. FIG. 1E is a full frontal perspective view of the device independent of a head, where 1 and 4 represent the left and right sides, respectively, of a headband in two halves fastened together by an adjustable clip, snap closure, velcro, or other such means, 5, said headband connected to a left and right hook, 2 and 3, respectively, positioned at and thereby raising the opposing corners of the lips.

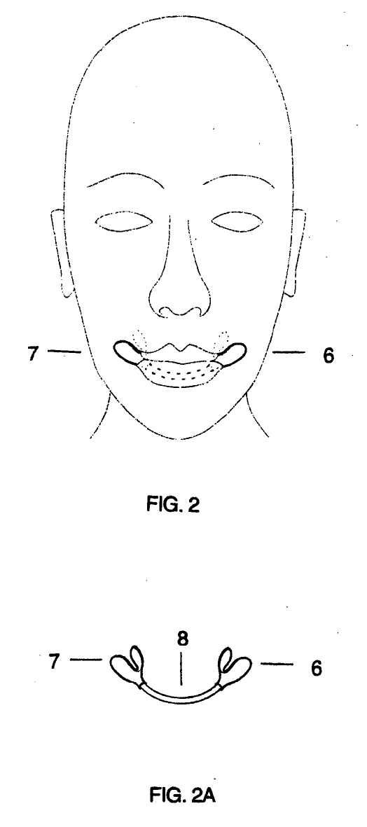

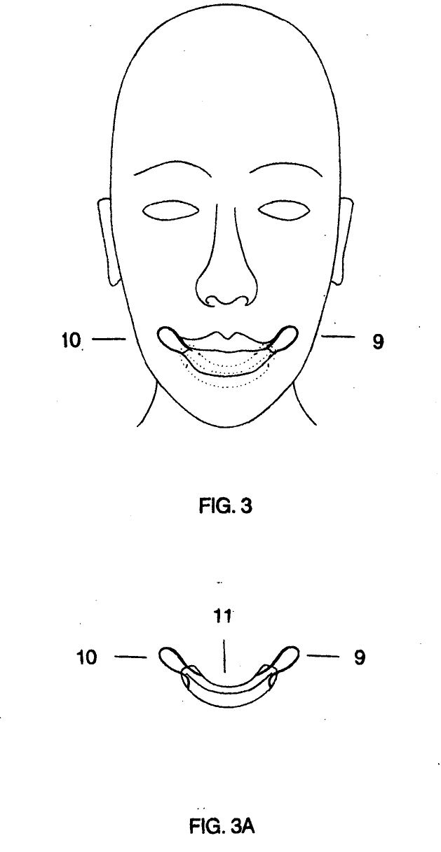

FIG. 2 is a frontal perspective view of the device on a head, showing the outside right and left portions, 6 and 7, respectively, of two removable saddles capped to the ends of an upwardly curved rod inside the lower lip, positioned at and thereby raising the lateral extremities of the lips. FIG. 2A is a frontal perspective view of the device alone, depicting two removable right and left saddle caps, 6 and 7, respectively, positioned at the opposing corners of the lips, and attached to the ends of an upwardly curved rod, 8, which fits inside the lower lip, thereby raising the lateral extremities of the lips.

FIG. 3 is a frontal perspective view of the device on a head, portraying the ends of two upward and outwardly protruding right and left adjustable spurs, 9 and 10, respectively, attached to a flexible lower dental plate, which can be positioned at and thereby raise the opposing corners of the lips. FIG. 3A is a frontal perspective view of the device by itself, illustrating a flexible lower dental plate, 11, with upward and outwardly protruding right and left adjustable spurs, 9 and 10, respectively, which can be positioned at either end of said plate to raise the opposing corners of the lips.

Those versed in the art will appreciate that changes and emendations can be made to the embodiments and descriptions herein without departing from the spirit of the present innovation. All such alternatives and alterations are intended to be subsumed by the aforestated claims.

I claim:

1. A method for improving mood by physically elevating both lateral extremities of the lips with a corresponding alteration of the facial muscles that transmit by nerves said informational change to appropriate centers in the brain which then invoke the states of pleasure, elation, or happiness.

2. The means of claim 1 wherein, for the purpose of raising both lateral extremities of the lips, hooks of noninjurious surfaces and nontoxic materials are pulled into an upward position by a stretchable or otherwise adjustable headband or earpieces of various design and composition.

3. The means of claim 1 wherein a semirigid rod, of noninjurious shape and surface as well as nontoxic material, curving upward and which can be fixed in size or cut to accommodate the mouth dimensions of the user, fitting inside the lower lip in front of the teeth, utilizes fixed or detachable saddles, also of noninjurious surfaces and nontoxic material, at its ends to effect the purpose of lifting both lips at their lateral extremities.

4. The means of claim 1 wherein a flexible lower dental plate, of noninjurious shape and surface as well as nontoxic material, fitting over the teeth, with upwardly protruding fixed or adjustable spurs positioned at both ends of the lips, is employed to elevate said lip extremities.
